(19) United States
(12) Patent Application Publication
(10) Pub. No.: US 2024/0324652 A1
(43) Pub. Date: Oct. 3, 2024

(54) METHODS OF PREPARING COMPOSITIONS COMPRISING A CONSTITUENT, DERIVATIVE OR EXTRACT OF CANNABIS

(71) Applicant: Nicoventures Trading Limited, London (GB)

(72) Inventors: Steven ALDERMAN, Winston-Salem, NC (US); Thomas POOLE, Winston-Salem, NC (US); Michael DANIEL, Winston-Salem, NC (US); Kai TANG, Winston-Salem, NC (US); Keyi XU, Winston-Salem, NC (US); Karina MCQUILLAN, London (GB)

(21) Appl. No.: 18/579,797

(22) PCT Filed: Jul. 21, 2022
(86) PCT No.: PCT/GB2022/051901

§ 371 Date: Jan. 16, 2024

Related U.S. Application Data
(60) Provisional application No. 63/224,565, filed on Jul. 22, 2021.

(51) Int. Cl. A24B 15/16; A24B 13/00

(52) U.S. Cl. CPC A24B 15/16; A24B 13/00

(57) ABSTRACT

The present invention relates to methods for preparing compositions comprising a constituent, derivative or extract of cannabis. The invention also relates to the compositions and products incorporating them.

### TECHNICAL FIELD

The present invention relates to methods for preparing compositions comprising a constituent, derivative or extract of cannabis. The invention also relates to the compositions and products incorporating them.

### BACKGROUND

Oral products comprising cannabinoids are known and seek to deliver the cannabinoids to the user.

### SUMMARY

According to a first aspect of the present invention, there is provided a method for preparing a composition comprising one or more constituent, derivative or extract of cannabis in non-crystalline form, the method comprising extruding a precursor composition comprising one or more constituent, derivative or extract of cannabis.

In some embodiments, the extruded composition is soluble or dispersible in water.

In some embodiments, the one or more constituent, derivative or extract of cannabis in the extruded composition is soluble in water.

In some embodiments, the constituent, derivative or extract of cannabis is one or more compounds selected from: cannabinoids; terpenes; alkaloids; and flavonoids.

In some embodiments, the constituent, derivative or extract of cannabis is selected from the group consisting of: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabmolic acid (THCA), and tetrahydrocannabivarinic acid (THCV A).

In some embodiments, the precursor composition comprises one or more acid form cannabinoid.

In some embodiments, the acid form cannabinoid is selected from the group consisting of cannabidiolic acid (CBDA), tetrahydrocannabinolic acid (THCA).

In some embodiments, the precursor composition comprises one or more constituent, derivative or extract of cannabis in solution.

In some embodiments, the precursor composition comprises one or more constituent, derivative or extract of cannabis in a colloid.

In some embodiments, the precursor composition comprises one or more constituent, derivative or extract of cannabis in solid form.

In some embodiments, the precursor composition comprises the one or more constituent, derivative or extract of cannabis in crystalline form.

In some embodiments, the extruded composition further comprises a water soluble material.

In some embodiments, the water soluble material is selected from the group consisting of: sugar alcohols; disaccharides; polysaccharides, such as cellulose, starch and their derivatives; dextrins; dextrates; natural gums; and polymers and copolymers, such as cellulosics.

In some embodiments, the water soluble material is selected from the group consisting of: mannitol, sorbitol, xylitol, isomalt, erythritol, arabitol, ribitol, maltitol, dulcitol, iditol and lactitol guar gum, acacia gum (also known as gum arabic), xanthan gum, locust bean gum, gellan gum, alginates and sodium alginates, hydroxypropyl methylcellulose (HPMC), hydroxypropyl methylcellulose acetate succinate (HPMCAS), polyvinylpyrrolidone (PVP), polyethylene glycol (PEG), polyethylene oxide (PEO), Macrogol 15 Hydroxystearate (Solutol HS 15®), and Vitamin E Polyethylene Glycol Succinate (Vit E TPGS).

In some embodiments, the water soluble material is present in an amount of from about 10 to about 70% by weight, based on the total weight of the extruded composition.

In some embodiments, the constituent, derivative or extract of cannabis is present in an amount of from about 0.1 to about 30% by weight, based on the total weight of the extruded composition.

In some embodiments, the precursor composition comprises an additive which slows or inhibits crystallisation of the one or more cannabinoid in an aqueous environment.

In some embodiments, the additive is selected from the group consisting of polyvinylpyrrolidone (PVP), hydroxypropyl cellulose (HPC), hydroxypropyl methylcellulose (HPMC) and carboxymethyl cellulose (CMC).

In some embodiments, the precursor composition further comprises a surfactant.

In some embodiments, the surfactant is selected from the group consisting of: glyceryl monooleate; and sodium lauryl sulfate (sodium dodecyl sulfate, SLS, or SDS), docusate sodium, lecithin, polyoxyethylene sorbitan fatty acid esters (Polysorbate, Tween®), polyoxyethylene 15 hydroxy stearate (Macrogol 15 hydroxy stearate, Solutol HS15®), polyoxyethylene castor oil derivatives (Cremophor® EL, ELP, RH 40), polyoxyethylene stearates (Myrj®), sorbitan fatty acid esters (Span®), polyoxyethylene alkyl ethers (Brij®)), polyoxyethylene nonylphenol ether (Nonoxynol®), sugar esters and lecithins.

In some embodiments, the precursor composition comprises surfactant in an amount of from about 0.5 to about 10% by weight, based on the total weight of the composition.

In some embodiments, the extruded composition further comprises a disintegrant selected from the group consisting of: croscarmellose, sodium starch glycolate, and crospovidone, povidone (PVP); and the like.

In some embodiments, the extruded composition further comprises an effervescent agent or combination of agents.

In some embodiments, the extruded composition comprises a flavour or sensate.

In some embodiments, the extruded composition comprises a further active agent.

In some embodiments, the precursor composition comprising a cannabis plant extract.

In some embodiments, the cannabis plant is hemp.

In some embodiments, the extruded composition is formed into a solid unit dosage form, a powder or granules.

In some embodiments, the extruded composition is formed into particles having a volume mean particle size of from about 50 μm to about 500 μm.

In some embodiments, the method comprises cutting and/or spheronisation of the extruded composition.

According to a second aspect of the present invention, there is provided an extruded composition obtained or obtainable by a method according to the first aspect.

According to a third aspect, there is provided an oral product for providing buccal delivery of one or more constituent, derivative or extract of cannabis, comprising a composition obtained or obtainable by a method according to the first aspect.

In some embodiments, the release of one or more constituent, derivative or extract of cannabis from the oral product begins within a period of 5, 10, 15 or 30 seconds following exposure to the aqueous environment of the oral cavity.

In some embodiments, the one or more constituent, derivative or extract of cannabis is released from the oral product over a period of at least 5, 10, 15, 20, 25, 30, 45 or 60 minutes following exposure to the aqueous environment of the oral cavity.

In some embodiments, the oral product comprises a pouch containing the extruded composition.

In some embodiments, the oral product comprises a dissolving strip comprising the extruded composition.

In some embodiments, the dissolving strip comprising a bioadhesive.

### BRIEF DESCRIPTION OF THE DRAWINGS

Embodiments of the invention will now be described, by way of example only, with reference to accompanying drawing, in which:

FIG. 1 is a perspective view illustrating a pouched product according to an embodiment of the present disclosure.

### DETAILED DESCRIPTION

The present invention seeks to provide methods for preparing compositions comprising one or more constituent, derivative or extract of cannabis. In particular embodiments, the methods provide compositions that include the one or more constituent, derivative or extract of cannabis in a form that will be readily released from the composition when exposed to an aqueous environment, such as in the oral cavity.

The compositions are intended for human use. They may also be configured for oral use and deliver the constituent, derivative or extract of cannabis, as well as optionally other substances such as flavours and/or active ingredients during use.

In some embodiments, the constituent, derivative or extract of cannabis is a cannabinoid.

Cannabinoids are lipid-soluble, hydrophilic molecules that are insoluble or poorly soluble in water. As such, delivery of cannabinoids can be challenging as it is difficult to solubilise them into a form suitable for release from the dosage form and for absorption. Even when solubilised, cannabinoids are quick to recrystallize when in an aqueous environment, which significantly impairs their bioavailability and thus their efficacy.

According to the present invention, the solubility of the one or more constituent, derivative or extract of cannabis is enhanced by including them in a precursor composition that is extruded in order to prepare a composition. The extrusion of the precursor composition will disperse the constituent, derivative or extract of cannabis within the composition. It may also cause physical and/or chemical changes to the one or more constituent, derivative or extract of cannabis, which may enhance its solubility.

In some embodiments, the precursor composition also includes other components such as a water soluble material which may form a matrix in the extruded composition. Provision of the constituent, derivative or extract of cannabis distributed within a water soluble matrix has been found to enhance the release of the constituent, derivative or extract of cannabis on exposure to an aqueous environment, and releases the constituent, derivative or extract of cannabis in a form that may be readily absorbed through the mucosa and into the bloodstream.

Extrusion Extrusion involves the feeding of the precursor composition through an orifice to produce an extruded agglomerate. The process applies pressure to the precursor composition combined with shear forces. In some embodiments, the extrusion process also involves elevated temperatures.

Extrusion may be performed using one of the main classes of extruders: screw, sieve and basket, roll, ram and pin barrel extruders. A single screw or twin screw extruder may be used.

In some embodiments, the first step is to combine the components of the precursor composition into hopper. The precursor composition may include, for example, a dry polymer, selected from cellulosic/starch-based polymers/PVP/HPC, and an active component, such as crystals of a cannabinoid such as CBD.

The solid components may be pre-mixed. In some embodiments, the size of the particles of the solid ingredients prior to extrusion is no greater than about 1000 μm, or no greater than about 800 μm.

In some embodiments, during extrusion the precursor composition is exposed to elevated pressure and temperature and is forced though an orifice, such as a shaping nozzle or die, to form an extruded composition. In some embodiments, the extruded composition has a string, strand or rod-like form and/or may be cut into segments of a desired length as it exits the orifice or later to provide segments of desired length.

In some embodiments, the precursor composition is exposed to temperatures from about 40° C. to about 200° C., or from about 80° C. to about 130° C., or from about 60° C. to about 95° C. within the extruder. In some embodiments, including those using double extrusion, the precursor composition is exposed to temperatures from about 70° C. to about 95° C. within the extruder. In some embodiments, including those using single extrusion, the precursor composition is exposed to temperatures from about 60° C. to about 80° C. within the extruder. Individuals skilled in the art carefully balance component stability and temperature. It is often desirable to heat components to a molten state but for less stable components this is sometimes avoided.

The precursor composition may be exposed to pressures (immediately before the die or nozzle) ranging from about 2 bar to about 100 bar, or from about 5 bar to about 60 bar, depending on the design of the die or nozzle being used. Pressures significantly higher than 100 bar may also be used in some embodiments.

The application of heat, pressure and shear forces by the extrusion process causes crystalline solids, such as CBD and other constituents, derivatives or extracts of cannabis, to be converted into an amorphous form, as the extrusion process “breaks” the crystalline structure.

In some embodiments, the extrusion process is dry or solvent-free extrusion. Dry extrusion is a form of extrusion processing in which a dry or substantially dry mixture (the precursor composition) is extruded.

In the case of dry extrusion, no liquid (such as water) needs to be added into the screw system. During such extrusion, pressure is applied and the material is compacted together. Dry extrusion can rely solely on the heat generated from the friction of the material as it passes through the barrel by a screw. The heat generated can be enough to turn at least some of the ingredients into a molten state.

In some embodiments, the precursor composition is a dry or substantially dry mixture including the one or more constituent, derivative or extract of cannabis in solid form, as well as optionally other solid, particulate materials including, for example, water soluble matrix-forming materials.

Dry extrusion means that the extruded composition does not have to be heated to dry it, which saves energy, simplifies production and also avoids the potential loss of volatiles.

In some embodiments, the extrusion process is a hot melt extrusion process. The process involves the application of heat, pressure and agitation to mix materials together and force them through a die. High shear hot melt extruders blend materials and break up particles.

Hot melt extrusion may be carried out at a temperature above the recrystallization temperature of the one or more constituent, derivative or extract of cannabis, to keep the material in melted form, making it easier to push the material through the die and creating a homogenous and amorphous extruded composition. Hot melt extrusion can result in the molecular dispersion of the poorly soluble one or more constituent, derivative or extract of cannabis within the extruded composition.

In some embodiments, liquids may be added to the precursor composition to be extruded, for a wet extrusion process. For example, in some embodiments, water may be added to the precursor composition, for example as a processing aid to assist dissolution or solubilisation of components of the composition, to improve the homogeneity of the resultant extruded composition or to aid binding or agglomeration.

For example, any water soluble matrix-forming materials may be dissolved in or solubilised by the presence of water.

As discussed above, many constituents, derivatives or extracts of cannabis are lipophilic and will not dissolve in water. Therefore, other liquid components may be included in the precursor composition if these constituents, derivatives or extracts are to be dissolved or solubilised. Suitable solvents include, for example: benzyl alcohol, mineral oil; polyethylene glycol (PEG); propylene glycol; and triacetin.

In some embodiments, wetting agents or surfactants may be included in the precursor composition.

In some embodiments, the ratio of water soluble matrix material to liquid solvent, surfactant or plasticizer, if included, should be around 80:20. Also, if used, a liquid component should be selected that is compatible with both the one or more constituent, derivative or extract of cannabis and the water soluble matrix material used.

The extruded agglomerate will be shaped by the nozzle or die through which it is forced. In some embodiments, the extruded agglomerate is cut into pieces of desired length. The pieces formed in this way may be used directly, for example in an oral product as described herein. Alternatively, the pieces may undergo further processing.

In some embodiments, the nozzle or die is shaped to provide a solid strand of extruded composition. For example, the extruded composition may have the form of a solid cylindrical rod. Alternatively, the extruded composition may have different cross-sectional shapes, including oval, polygonal (such as triangular, square, etc.), and stars.

In some embodiments, the extruded composition is formed into a desired shape selected to enhance or promote the release of the one or more constituent, derivative or extract of cannabis, for example by providing a form having a large surface area per unit volume. This large surface area may be provided on the outer surface of the extruded composition, for example by selecting cross-sectional shapes with large perimeter. Alternatively or in addition, the large surface area may be provided through the creation of channels within the extruded composition.

In some embodiments, the nozzle is shaped to provide an extruded agglomerate with inner channels. These inner channels provide further surface area and can enhance release of the one or more constituent, derivative or extract of cannabis, and other component of the composition.

By means of various nozzle or die designs and/or different process parameters within the extruder, including the temperature, pressure and shear forces, extruded compositions with different physical properties may be prepared, including different dissolution and release properties.

In some embodiments, the extruded composition is shaped upon discharge from the extruder. In some embodiments, the extruded composition is cut to an initial length, for example 1 metre, and allowed to cool before then being cut into sections of the desired length to provide pieces or particles of the desired dimensions.

In some embodiments, the extruded composition may be cooled just before or just as it leaves the extruder. In some embodiments, the cooling is intensive and involves exposing the extruded composition, which will be at an elevated temperature, for example from about 30° C. to about 100° C., or from about 40° C. to about 70° C., to a cooling means that will reduce the temperature to within a range of from about 0° C. to about 70° C., from about 0° C. to about 50° C., from about 5° C. to about 25° C. or from about 5° C. to about 15° C. This rapid cooling of the extruded composition may enhance the internal and external stability of the extruded composition. In some embodiments, it is the nozzle or die that is cooled to achieve this effect.

In some embodiments, it may be desirable to control the temperature of the precursor composition during extrusion, including before feeding the composition through the nozzle or die. This is especially the case where the precursor composition includes temperature sensitive components. Thus, in some embodiments, extrusion of the precursor composition includes reducing the temperature of the precursor composition before it reaches the nozzle or die. Such cooling of the precursor composition may result in the formation of an extruded composition with beneficial properties, or may improve the strand-shaping process, for example where channels are to be formed within the extruded strand.

In some embodiments, spheronisation is used to further process extruded segments or pellets. These segments are typically cylindrically-shaped and are cut or broken into uniform lengths following extrusion. They are then gradually transformed into spherical shapes by spheronisation. This shaping occurs as a result of plastic deformation and spheronisation can lead to spherical agglomerated structures, often with a nearly uniform diameter.

In some embodiments, the extruded composition will be milled and/or spheronised to produce particles that will flow freely, for example to allow them to be fed into a pouch. The optimal particle size distribution for good flowability has an approximate d90 of 500 μm or 200 μm.

### Constituent, Derivative or Extract of Cannabis

As used herein, any compound or mixture of compounds which may be obtained from cannabis may be a constituent, derivative or extract thereof, including synthetic versions of such compound(s) or such compound(s) derived from other natural sources.

In some embodiments the constituent, derivative or extract of cannabis comprises, or is, one or more compounds selected from: cannabinoids (such as phytocannabinoids that may optionally be THC and/or CBD); terpenes (such as triterpenes); alkaloids; and flavonoids.

In some embodiments the constituent, derivative or extract of cannabis comprises one or more compounds selected from: cannabinoids (such as phytocannabinoids) and terpenes (such as triterpenes).

In some embodiments the constituent, derivative or extract of cannabis comprises one or more cannabinoids, such as phytocannabinoids.

Cannabinoids are a class of natural or synthetic chemical compounds which act on cannabinoid receptors (i.e., CB1 and CB2) in cells that repress neurotransmitter release in the brain. Cannabinoids may be naturally occurring (phytocannabinoids) from plants such as cannabis, from animals (endocannabinoids), or artificially manufactured (synthetic cannabinoids). Cannabis species express at least 85 different phytocannabinoids, and are divided into subclasses, including cannabigerols, cannabichromenes, cannabidiols, tetrahydrocannabinols, cannabinols and cannabinodiols, and other cannabinoids. Cannabinoids found in cannabis include, without limitation: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabmolic acid (THCA), and tetrahydrocannabivarinic acid (THCV A).

In some embodiments, the cannabinoids are phytocannabinoids.

In some embodiments, the terpenes are triterpenes.

In particular embodiments, the constituent, derivative or extract of cannabis comprises, or is, tetrahydrocannabinol (THC) and/or cannabidiol (CBD).

In some embodiments, the constituent, derivative or extract of cannabis comprises, or is, THC.

In particular embodiments, the constituent, derivative or extract of cannabis comprises, or is, CBD.

In some embodiments, the constituent, derivative or extract of cannabis is present in an amount of from about 0.1 to about 30% by weight, based on the total weight of the composition. In some embodiments, the constituent, derivative or extract of cannabis is present in the composition in an amount of at least about 0.1%, at least about 0.2%, at least about 0.3%, at least about 0.4%, at least about 0.5%, at least about 0.6%, at least about 0.7%, at least about 0.8%, or at least about 0.9%. In some embodiments, the constituent, derivative or extract of cannabis is present in the precursor composition and/or the extruded composition in an amount of no more than about 1%, about 2%, about 3%, about 4%, about 5%, about 6%, about 7%, about 8%, about 9%, about 10%, about 15%, about 20%, or no more than about 30% by weight, based on the total weight of the composition.

### Water Soluble Matrix Materials

The water soluble matrix comprises a water soluble material within which the constituent, derivative or extract of cannabis is dispersed.

In some embodiments, the water soluble material is selected to be rapidly dissolved upon contact with an aqueous medium, for example, in the oral cavity. Examples of such rapidly dissolving materials include: sugar alcohols, such as mannitol, sorbitol, xylitol, isomalt, erythritol, arabitol, ribitol, maltitol, dulcitol, iditol and lactitol; disaccharides, such as sucrose, lactose and maltose; polysaccharides, such as cellulose, starch and their derivatives; dextrins, such as maltodextrin; dextrates; natural gums, such as guar gum, acacia gum (also known as gum arabic), xanthan gum, locust bean gum, gellan gum, alginates and sodium alginates; and polymers and copolymers, such as cellulosics (e.g., hydroxypropyl methylcellulose (HPMC), hydroxypropyl methylcellulose acetate succinate (HPMCAS)), polyvinylpyrrolidone (PVP), polyethylene glycol (PEG), and polyethylene oxide (PEO); Macrogol 15 Hydroxystearate (Solutol HS 15®); and Vitamin E Polyethylene Glycol Succinate (Vit E TPGS).

In some embodiments, the water soluble matrix material is selected from the group consisting of: Macrogol 15 Hydroxystearate (Solutol HS 15®); Polyethylene Oxide (PEO); Vitamin E Polyethylene Glycol Succinate (Vit E TPGS); isomalt; maltitol; mannitol; dextrates; dextrose; and erythritol.

In some embodiments, the precursor composition and/or the extruded composition comprises the water soluble material in an amount of from about 30 to about 70% by weight, based on the total weight of the composition.

The properties of the water soluble material affect the rate of release of the constituent, derivative or extract of cannabis from the matrix within which they are dispersed or embedding through factors including diffusion, permeation, and dissolution.

For the constituent, derivative or extract of cannabis to be released from the extruded composition, it must first be dissolved. Therefore, the ability of the constituent, derivative or extract of cannabis to dissolve and diffuse out of the matrix and into the surrounding aqueous environment relates directly to the rate at which water penetrates the matrix.

As the water soluble matrix dissolves in an aqueous environment, the constituent, derivative or extract of cannabis is released and may be absorbed. Release occurs as a result of the diffusion, which occurs as the constituent, derivative or extract of cannabis moves from a region of high concentration inside the matrix to a region of low concentration, namely the surrounding environment.

In some embodiments, diffusion and release of the constituent, derivative or extract of cannabis may be controlled by including in the matrix a hydrophilic polymer material that hydrates on contact with an aqueous liquid to form a gel layer at the matrix surface. This gel layer acts as a barrier to diffusion of the constituent, derivative or extract of cannabis out of the matrix. The gel layer also prevents additional water from entering the matrix too rapidly. Over time, water gradually permeates into the interior of the matrix, increasing the gel layer and gradually the gel will dissolve from the outside, releasing the constituent, derivative or extract of cannabis. Such a matrix is useful in embodiments where delayed or sustained release of the constituent, derivative or extract of cannabis is desired.

For constituent, derivative or extract of cannabis of low water solubility, their release is primarily controlled by the rate at which the matrix hydrates and dissolves in the aqueous environment. Therefore, the hydration rate and gel layer properties of the hydrophilic polymer largely control the release characteristics.

In some embodiments, the matrix comprises hydroxypropyl methylcellulose (HPMC) as a hydrophilic polymer for providing modified-release of the constituent, derivative or extract of cannabis. The molecular weight of the HMPC used can be selected to provide the desired gel layer thickness and gel layer growth. In some embodiments, an HPMC is selected that quickly hydrates to form a gelatinous layer that surrounds the matrix and controls ingress of water and disintegration of the matrix. The rate of hydration of HPMC is related to the proportion of hydroxypropyl and methoxyl substitution on the polymer chains, and is also influenced by molecular weight.

Natural gums such as sodium alginate, xanthan gum, locust bean gum and guar gum may also be used as hydrophilic matrices as they have the ability to quickly hydrate and swell in water to form a gel layer. Polysaccharides may also be used to produce a strong gel.

### Matrix Disintegration Additives

In some embodiments, the water soluble matrix may comprise one or more additives that enhance disintegration and thereby improve the release and bioavailability of the constituent, derivative or extract of cannabis. Such additives may be materials that instantaneously dissolve on contact with an aqueous environment, providing rapid disintegration of the matrix and enhancing the dissolution and bioavailability of the constituent, derivative or extract of cannabis.

In some cases, the disintegration additive may aid the rapid disintegration of the matrix due to the rapid uptake of water from the medium, swelling, and burst effect.

Examples of suitable disintegrants include: croscarmellose, sodium starch glycolate, and crospovidone, povidone (PVP); and the like.

Effervescent agents, such as sodium bicarbonate in conjunction with an organic acid such as citric or tartaric acid may also act to enhance disintegration of the matrix. Contact with an aqueous medium causes effervescence which affects the structure of the matrix, assisting disintegration and release of the constituent, derivative or extract of cannabis.

Matrix disintegration additives such as croscarmellose can be included in the matrix in an amount of from about 0.5% to about 8% by weight, based on the total weight of the composition. Effervescent agents will generally need to be included in a greater amounts. For example, the acid may be included in an amount from about 5% to 20%, with the bicarbonate present in an amount from about 5% to 20% (and dependent on the amount of acid), by weight, based on the total weight of the composition.

### Surfactants

In some embodiments, the matrix further comprises one or more surfactant or wetting agent. One function of the surfactant may be to allow the constituent, derivative or extract of cannabis to remain in a lipophilic droplet when exposed to an aqueous environment. This means that the constituent, derivative or extract of cannabis is in a form in which it may be absorbed from the oral cavity via the mucosa.

The surfactants, when used appropriately, can create an emulsion with the aqueous environment. Emulsions are evenly dispersed oil droplets within an aqueous environment. Such an emulsion enables the constituent, derivative or extract of cannabis to remain in the dispersed oil droplets and the surfactant is making the aqueous environment more acceptable for the lipophilic compounds.

Examples of suitable surfactants include: glyceryl monooleate; and sodium lauryl sulfate (sodium dodecyl sulfate, SLS, or SDS), docusate sodium, lecithin, polyoxyethylene sorbitan fatty acid esters (Polysorbate, Tween®)), polyoxyethylene 15 hydroxy stearate (Macrogol 15 hydroxy stearate, Solutol HS15®), polyoxyethylene castor oil derivatives (Cremophor® EL, ELP, RH 40), polyoxyethylene stearates (Myrj®), sorbitan fatty acid esters (Span®), polyoxyethylene alkyl ethers (Brij®), polyoxyethylene nonylphenol ether (Nonoxynol®), sugar esters and lecithins.

In some embodiments, the surfactant is present in an amount of from about 0.5 to about 10% by weight, based on the total weight of the composition.

### Other Components

Further ingredients, which could include flowability enhancers, flavourants, additional actives, sweeteners, and bulk carriers, etc., may be incorporated into the extruded composition or may be separately incorporated into the oral product.

In some embodiments, the extruded composition may include one or more further functional materials. These functional materials may comprise one or more of pH regulators, colouring agents, preservatives, binders, fillers, stabilizers, and/or antioxidants.

In some embodiments, binding agents are included in the composition that aid in producing a pleasant mouth feel. Suitable binding agents include, for example, microcrystalline cellulose.

In some embodiments, the extruded composition may include one or more diluent.

Suitable diluents include, for example: calcium carbonate; disodium hydrogen phosphate; lactose (hydrous, anhydrous, monohydrate or spray dried); and magnesium oxide.

In some embodiments, the extruded composition may include one or more antimicrobial preservative. Suitable preservatives include, for example: benzyl alcohol, cetylpyridine chloride; glycerin; methyl paraben; propylene glycol; propylene paraben; potassium sorbate; sodium benzoate; sorbic acid; and sodium propionate.

In some embodiments, the extruded composition may include one or more solvent, optionally wherein the solvent dissolves or solubilises one or more constituent, derivative or extract of cannabis. Suitable solvents include any that are capable of solvating moderately lipophilic components such as terpenes, and highly lipophilic components such as cannabinoids. For example, suitable solvents may include: benzyl alcohol, mineral oil; polyethylene glycol (PEG); propylene glycol; glycerine; ethanol; and triacetin.

In some embodiments, the extruded composition may include one or more mucoadhesive. Suitable mucoadhesives include, for example: polyethylene oxide, proteins such as gelatin; and carbohydrates such as starch and disaccharides; and polysaccharide polymers such as amylopectin, pullulan, hyaluronic acid and tamarind xyloglucan.

In some embodiments, the extruded composition may include one or more pH modifiers. Suitable pH modifiers include, for example: citric acid or sodium acetate.

Cyclodextrins form another class of functional excipients that are widely used to enhance solubility of the constituent, derivative or extract of cannabis. Cyclodextrins are cyclic oligosaccharides which comprise glucopyranose units and which have a lipophilic central cavity and an outer hydrophilic shell. Thus, the cyclodextrin is able to form water-soluble inclusion complexes with the poorly soluble constituent, derivative or extract of cannabis. Formulation with cyclodextrins may also improve the physical and chemical stability of some constituents, derivatives or extracts of cannabis.

Cyclodextrin and cyclodextrin derivatives which may be useful in the present invention include α-cyclodextrin, β-cyclodextrin, γ-cyclodextrin, hydroxypropyl-β-cyclodextrin, dimethyl-β-cyclodextrin, sulphobutylether cyclodextrin, 2,6-dimethyl-β-cyclodextrin, 2,3,6-trimethyl-β-cyclodextrin.

In some embodiments, the extruded composition may comprise a hydrophobic component that does not dissolve and forms a porous scaffold that does not swell, dissolve or erode over time in an aqueous environment. The constituent, derivative or extract of cannabis is released from such a matrix as the water diffuses through the porous matrix and makes contact with embedded constituent, derivative or extract of cannabis which then diffuses out through the pores of the matrix. In some embodiments, the matrix comprises both a hydrophobic component and a water-soluble component that will diffuse out rapidly in an aqueous environment producing a matrix with increased porosity.

Examples of water-insoluble matrix polymers include PVA with polyvinylpyrrolidone, and ammonium methacrylate copolymer.

In some embodiments, the extruded composition may include one or more porous materials. Suitable porous materials include, for example, mesoporous silica grades that may be included to hold poorly-soluble actives in the amorphous form, thus enhancing solubility of those actives.

### Oral Products

The extruded compositions provided herein are intended for oral use. This means that the composition is provided in a form such that during use, saliva in the mouth of the user causes one or more of the components of the composition to pass into the mouth of the user.

In some embodiments, the composition is adapted to deliver components to a user through mucous membranes in the user's mouth, the user's digestive system, or both. In some embodiments, the composition is configured to deliver the one or more constituent, derivative or extract of cannabis, so that it can be absorbed through the mucous membranes in the mouth or absorbed through the digestive tract when the product is used. In some embodiments, the majority of the constituent, derivative or extract of cannabis is adsorbed through the mucous membranes of the mouth.

The compositions as disclosed herein can be formed into a variety of shapes, including pills, tablets, spheres, strips, films, sheets, coins, cubes, beads, ovoids, obloids, cylinders, bean shaped, sticks, or rods. Cross-sectional shapes of the composition can vary, and example cross-sectional shapes include circles, squares, ovals, rectangles, and the like. Such shapes can be formed in a variety of manners using equipment such as moving belts, nips, extruders, granulation devices, compaction devices, and the like.

It is important to note that larger particles of the same composition will take longer to dissolve than smaller particles, due to the smaller surface area to volume ratio. Therefore, the release of the one or more constituent, derivative or extract of cannabis can be controlled by selecting particles of a particular size or particles of different sizes in appropriate proportions. For example, the larger particles could be around 500 μm while the smaller beads could be between 100 and 50 μm. If the intention is to have a short or instant release, then it may be desirable to keep the d90 to between 125 and 150 μm. Flowability will start to become problematic if the d90 drops to 50 μm and a flowability aid will need to be added to the beads. The size of particles as referred to herein may be measured by sieving.

In some embodiments, the oral product may be in a form such as a gel capsule, a chew, a pastille, a lozenge, a strip, a powder or a chewing gum. Such formulations are well known to one skilled in the art.

### Pouched Products

In some embodiments, the extruded composition is provided in a pouch. In such embodiments, a portion of the composition is provided sealed within a wrapping material. The wrapping material may be a non-woven fleece.

The composition within the pouch may have the form of a single, monolithic portion, or may comprise a plurality of smaller portions, such as granules or beads, or the like.

Where the composition is to be used in a pouched product, the water soluble matrix is preferably highly water soluble or water dispersible, even if the pouch itself is fully dissolvable. This way the water soluble matrix will dissolve in the saliva and release the constituent, derivative or extract of cannabis to then be absorbed by the cheek or gum tissue.

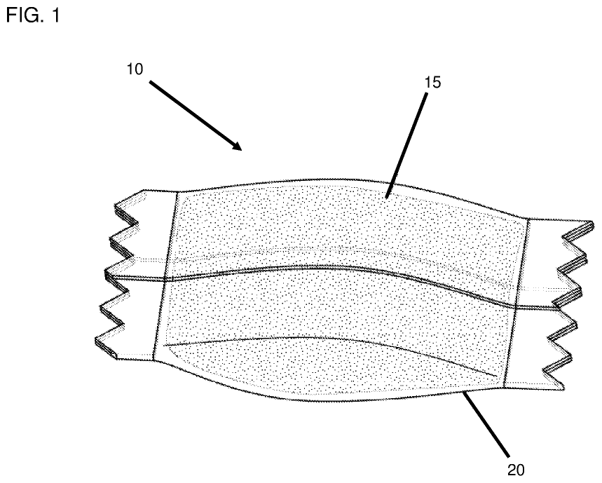

An example of a pouched product 10 is illustrated in FIG. 1. In the illustrated embodiments, the pouch comprises an outer water-permeable container 20 in the form of a pouch which contains a particulate mixture 15 comprising the water soluble matrix described herein. The orientation, size, and type of outer water-permeable pouch and the type and nature of the composition contained therein that are illustrated are examples only and are not to be construed as limiting.

### Dissolvable Products

In some embodiments, there are provided dissolvable products configured for oral use, the products comprising extruded compositions comprising at least one constituent, derivative or extract of cannabis. For example, the product may be a dissolvable strip which is placed in the oral cavity of the user and then rapidly dissolves on contact with the aqueous environment, releasing the constituent, derivative or extract of cannabis. The dissolvable product may comprise a mucoadhesive to hold it in position on a mucosal membrane, to ensure that release and absorption of the constituent, derivative or extract of cannabis is occurs at or near that location, encouraging absorption through the mucosal membrane.

### Dissolution Properties of Composition

In some embodiments, the compositions start to release of a constituent, derivative or extract of cannabis within a period of as little as 5, 10, 15 or 30 seconds following exposure of the composition to water. In some embodiments, this exposure may be in the aqueous environment of the oral cavity.

In some embodiments, release of constituent, derivative or extract of cannabis continues over a period of at least 5, 10, 15, 20, 25, 30, 45 or 60 minutes following exposure water. Once again, the exposure to water may be as a result of exposure to the aqueous environment when the composition is placed in the oral cavity.

In some embodiments, an oral product starts to release of a constituent, derivative or extract of cannabis within a period of as little as 5, 10, 15 or 30 seconds following exposure of the oral product to water. In some embodiments, this exposure may be in the aqueous environment of the oral cavity.

In some embodiments, release of constituent, derivative or extract of cannabis from an oral product continues over a period of at least 5, 10, 15, 20, 25, 30, 45 or 60 minutes following exposure of the oral product to water. Once again, the exposure to water may be as a result of exposure of the oral product to the aqueous environment when placed in the oral cavity.

### Additional Actives

In some embodiments, the extruded composition comprises one or more active substance in addition to the one or more constituent, derivative or extract of cannabis.

The further active substance as used herein may be a physiologically active material, which is a material intended to achieve or enhance a physiological response. The active substance may for example be selected from nutraceuticals, nootropics, and psychoactives. The active substance may be naturally occurring or synthetically obtained.

In some embodiments, the one or more additional active ingredients, may include, for example: botanical ingredients, stimulants, amino acids, nicotine components, pharmaceutical ingredients, nutraceutical ingredients, medicinal ingredients, terpenes, and combinations thereof.

In certain embodiments, the active ingredient is selected from the group consisting of caffeine, taurine, GABA, theanine, vitamin C, lemon balm extract, ginseng, citicoline, sunflower lecithin, and combinations thereof. For example, the active ingredient can include a combination of caffeine, theanine, and optionally ginseng. In another embodiment, the active ingredient includes a combination of theanine, gamma-amino butyric acid (GABA), and lemon balm extract. In a further embodiment, the active ingredient includes theanine, theanine and tryptophan, or theanine and one or more B vitamins (e.g., vitamin B6 or B12). In a still further embodiment, the active ingredient includes a combination of caffeine, taurine, and vitamin C.

The particular percentages of active ingredients present will vary depending upon the desired characteristics of the particular product. Typically, an active ingredient or combination thereof is present in a total concentration of at least about 0.001% by weight of the composition, such as in a range from about 0.001% to about 20%. In some embodiments, the active ingredient or combination of active ingredients is present in a concentration from about 0.1% w/w to about 10% by weight, such as, e.g., from about 0.5% w/w to about 10%, from about 1% to about 10%, from about 1% to about 5% by weight, based on the total weight of the composition. In some embodiments, the active ingredient or combination of active ingredients is present in a concentration of from about 0.001%, about 0.01%, about 0.1%, or about 1%, up to about 20% by weight, such as, e.g., from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 25 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the composition. Further suitable ranges for specific active ingredients are provided below.

### Terpenes

Active ingredients suitable for use in the present disclosure can also be classified as terpenes, many of which are associated with biological effects, such as calming effects.

Terpenes are understood to have the general formula of (C₅H₈)n and include monoterpenes, sesquiterpenes, and diterpenes. Terpenes can be acyclic, monocyclic or bicyclic in structure.

Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

### Botanical

In some embodiments, the active ingredient comprises a botanical ingredient. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). For the purposes of the present disclosure, a “botanical” includes, but is not limited to, “herbal materials,” which refer to seed-producing plants that do not develop persistent woody tissue and are often valued for their medicinal or sensory characteristics (e.g., teas or tisanes). Reference to botanical material as “non-tobacco” is intended to exclude tobacco materials (i.e., does not include any Nicotiana species). In some embodiments, the compositions as disclosed herein can be characterized as free of any tobacco material (e.g., any embodiment as disclosed herein may be completely or substantially free of any tobacco material). By “substantially free” is meant that no tobacco material has been intentionally added. For example, certain embodiments can be characterized as having less than 0.001% by weight of tobacco, or less than 0.0001%, or even 0% by weight of tobacco.

When present, a botanical is typically at a concentration of from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the composition.

The botanical materials useful in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods”. Certain botanicals, as the plant material or an extract thereof, have found use in traditional herbal medicine, and are described further herein. Non-limiting examples of botanicals or botanical-derived materials include ashwagandha, Bacopa monniera, baobab, basil, Centella asiatica, Chai-hu, chamomile, cherry blossom, chlorophyll, cinnamon, citrus, cloves, cocoa, cordyceps, curcumin, damiana, Dorstenia arifolia, Dorstenia odorata, essential oils, eucalyptus, fennel, Galphimia glauca, ginger, Ginkgo biloba, ginseng (e.g., Panax ginseng), green tea, Griffonia simplicifolia, guarana, cannabis, hemp, hops, jasmine, Kaempferia parviflora (Thai ginseng), kava, lavender, lemon balm, lemongrass, licorice, lutein, maca, matcha, Nardostachys chinensis, oil-based extract of Viola odorata, peppermint, quercetin, resveratrol, Rhizoma gastrodiae, Rhodiola, rooibos, rose essential oil, rosemary, Sceletium tortuosum, Schisandra, Skullcap, spearmint extract, Spikenard, terpenes, tisanes, turmeric, Turnera aphrodisiaca, valerian, white mulberry, and Yerba mate.

In some embodiments, the active ingredient comprises lemon balm. Lemon balm (Melissa officinalis) is a mildly lemon-scented herb from the same family as mint (Lamiaceae). The herb is native to Europe, North Africa, and West Asia. The tea of lemon balm, as well as the essential oil and the extract, are used in traditional and alternative medicine. In some embodiments, the active ingredient comprises lemon balm extract. In some embodiments, the lemon balm extract is present in an amount of from about 1 to about 4% by weight, based on the total weight of the composition.

In some embodiments, the active ingredient comprises ginseng. Ginseng is the root of plants of the genus Panax, which are characterized by the presence of unique steroid saponin phytochemicals (ginsenosides) and gintonin. Ginseng finds use as a dietary supplement in energy drinks or herbal teas, and in traditional medicine. Cultivated species include Korean ginseng (P. ginseng), South China ginseng (P. notoginseng), and American ginseng (P. quinquefolius). American ginseng and Korean ginseng vary in the type and quantity of various ginsenosides present. In some embodiments, the ginseng is American ginseng or Korean ginseng. In specific embodiments, the active ingredient comprises Korean ginseng. In some embodiments, ginseng is present in an amount of from about 0.4 to about 0.6% by weight, based on the total weight of the composition.

### Stimulants

In some embodiments, the active ingredient comprises one or more stimulants. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis. In some embodiments, the active ingredient comprises caffeine. In some embodiments, the caffeine is present in an encapsulated form. One example of an encapsulated caffeine is Vitashure®, available from Balchem Corp., 52 Sunrise Park Road, New Hampton, NY, 10958.

When present, a stimulant or combination of stimulants (e.g., caffeine, theacrine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition. In some embodiments, the composition comprises caffeine in an amount of from about 1.5 to about 6% by weight, based on the total weight of the composition;

### Amino Acids

In some embodiments, the active ingredient comprises an amino acid. As used herein, the term “amino acid” refers to an organic compound that contains amine (—NH2) and carboxyl (—COOH) or sulfonic acid (SO3H) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-translational modification).

Non-limiting examples of non-proteinogenic amino acids include gamma-aminobutyric acid (GABA), taurine (2-aminoethanesulfonic acid), theanine (L-γ-glutamylethylamide), hydroxyproline, and beta-alanine. In some embodiments, the active ingredient comprises theanine. In some embodiments, the active ingredient comprises GABA. In some embodiments, the active ingredient comprises a combination of theanine and GABA. In some embodiments, the active ingredient is a combination of theanine, GABA, and lemon balm. In some embodiments, the active ingredient is a combination of caffeine, theanine, and ginseng. In some embodiments, the active ingredient comprises taurine. In some embodiments, the active ingredient is a combination of caffeine and taurine.

When present, an amino acid or combination of amino acids (e.g., theanine, GABA, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition.

### Vitamins and Minerals

In some embodiments, the active ingredient comprises a vitamin or combination of vitamins. As used herein, the term “vitamin” refers to an organic molecule (or related set of molecules) that is an essential micronutrient needed for the proper functioning of metabolism in a mammal. There are thirteen vitamins required by human metabolism, which are: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones). In some embodiments, the active ingredient comprises vitamin C. In some embodiments, the active ingredient is a combination of vitamin C, caffeine, and taurine.

When present, a vitamin or combination of vitamins (e.g., vitamin B6, vitamin B12, vitamin E, vitamin C, or a combination thereof) is typically at a concentration of from about 0.01% w/w to about 6% by weight, such as, e.g., from about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5%, about 0.6%, about 0.7%, about 0.8%, about 0.9%, about 1%, about 2%, about 3%, about 4%, about 5%, or about 6% by weight, based on the total weight of the composition.

In some embodiments, the active ingredient comprises a mineral or combination of minerals. As used herein, the term “mineral” refers to a chemical compound with a defined chemical composition and a specific crystal structure that occurs naturally in pure form. In some embodiments, the active ingredient comprises a magnesium-based mineral compounds, e.g., such as magnesium gluconate, magnesium citrate, and the like. When present, a mineral or combination of minerals (e.g., magnesium gluconate, magnesium citrate, or a combination thereof) is typically at a concentration of from about 0.01% w/w to about 6% by weight, such as, e.g., from about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5% about 0.6%, about 0.7%, about 0.8%, about 0.9%, about 1%, about 2%, about 3%, about 4%, about 5%, or about 6% by weight, based on the total weight of the composition.

### Antioxidants

In some embodiments, the active ingredient comprises one or more antioxidants. As used herein, the term “antioxidant” refers to a substance which prevents or suppresses oxidation by terminating free radical reactions, and may delay or prevent some types of cellular damage.

Antioxidants may be naturally occurring or synthetic. Naturally occurring antioxidants include those found in foods and botanical materials. Non-limiting examples of antioxidants include certain botanical materials, vitamins, polyphenols, and phenol derivatives.

Examples of botanical materials which are associated with antioxidant characteristics include without limitation acai berry, alfalfa, allspice, annatto seed, apricot oil, basil, bee balm, wild bergamot, black pepper, blueberries, borage seed oil, bugleweed, cacao, calamus root, catnip, catuaba, cayenne pepper, chaga mushroom, chervil, cinnamon, dark chocolate, potato peel, grape seed, ginseng, gingko biloba, Saint John's Wort, saw palmetto, green tea, black tea, black cohosh, cayenne, chamomile, cloves, cocoa powder, cranberry, dandelion, grapefruit, honeybush, echinacea, garlic, evening primrose, feverfew, ginger, goldenseal, hawthorn, hibiscus flower, jiaogulan, kava, lavender, licorice, marjoram, milk thistle, mints (menthe), oolong tea, beet root, orange, oregano, papaya, pennyroyal, peppermint, red clover, rooibos (red or green), rosehip, rosemary, sage, clary sage, savory, spearmint, spirulina, slippery elm bark, sorghum bran hi-tannin, sorghum grain hi-tannin, sumac bran, comfrey leaf and root, goji berries, gutu kola, thyme, turmeric, uva ursi, valerian, wild yam root, wintergreen, yacon root, yellow dock, yerba mate, yerba santa, Bacopa monniera, withania somnifera, Lion's mane, and silybum marianum. Such botanical materials may be provided in fresh or dry form, essential oils, or may be in the form of an extracts. The botanical materials (as well as their extracts) often include compounds from various classes known to provide antioxidant effects, such as minerals, vitamins, isoflavones, phytosterols, allyl sulfides, dithiolthiones, isothiocyanates, indoles, lignans, flavonoids, polyphenols, and carotenoids. Examples of compounds found in botanical extracts or oils include ascorbic acid, peanut endocarb, resveratrol, sulforaphane, beta-carotene, lycopene, lutein, co-enzyme Q, carnitine, quercetin, kaempferol, and the like. See, e.g., Santhosh et al., Phytomedicine, 12 (2005) 216-220, which is incorporated herein by reference.

Non-limiting examples of other suitable antioxidants include citric acid, Vitamin E or a derivative thereof, a tocopherol, epicatechol, epigallocatechol, epigallocatechol gallate, erythorbic acid, sodium erythorbate, 4-hexylresorcinol, theaflavin, theaflavin monogallate A or B, theaflavin digallate, phenolic acids, glycosides, quercitrin, isoquercitrin, hyperoside, polyphenols, catechols, resveratrols, oleuropein, butylated hydroxyanisole (BHA), butylated hydroxytoluene (BHT), tertiary butylhydroquinone (TBHQ), and combinations thereof.

When present, an antioxidant is typically at a concentration of from about 0.001% w/w to about 10% by weight, such as, e.g., from about 0.001%, about 0.005%, about 0.01% w/w, about 0.05%, about 0.1%, or about 0.5%, to about 1%, about 2%, about 3%, about 4%, about 5%, about 6%, about 7%, about 8%, about 9%, or about 10%, based on the total weight of the composition.

### Nicotine Component

In certain embodiments, the active ingredient comprises a nicotine component. By “nicotine component” is meant any suitable form of nicotine (e.g., free base or salt) for providing oral absorption of at least a portion of the nicotine present. Typically, the nicotine component is selected from the group consisting of nicotine free base and a nicotine salt. In some embodiments, the nicotine component is nicotine in its free base form, which easily can be adsorbed in for example, a microcrystalline cellulose material to form a microcrystalline cellulose-nicotine carrier complex. See, for example, the discussion of nicotine in free base form in US Pat. Pub. No. 2004/0191322 to Hansson, which is incorporated herein by reference.

In some embodiments, at least a portion of the nicotine component can be employed in the form of a salt. Salts of nicotine can be provided using the types of ingredients and techniques set forth in U.S. Pat. No. 2,033,909 to Cox et al. and Perfetti, Beitrage Tabakforschung Int., 12:43-54 (1983), which are incorporated herein by reference.

Additionally, salts of nicotine are available from sources such as Pfaltz and Bauer, Inc. and K&K Laboratories, Division of ICN Biochemicals, Inc. Typically, the nicotine component is selected from the group consisting of nicotine free base, a nicotine salt such as hydrochloride, dihydrochloride, monotartrate, bitartrate, sulfate, salicylate, and nicotine zinc chloride.

In some embodiments, at least a portion of the nicotine can be in the form of a resin complex of nicotine, where nicotine is bound in an ion-exchange resin, such as nicotine polacrilex, which is nicotine bound to, for example, a polymethacrylic acid, such as Amberlite IRP64, Purolite C115HMR, or Doshion P551. See, for example, U.S. Pat. No. 3,901,248 to Lichtneckert et al., which is incorporated herein by reference. Another example is a nicotine polyacrylic carbomer complex, such as with Carbopol 974P. In some embodiments, nicotine may be present in the form of a nicotine polyacrylic complex.

Typically, the nicotine component (calculated as the free base) when present, is in a concentration of at least about 0.001% by weight of the composition, such as in a range from about 0.001% to about 10%. In some embodiments, the nicotine component is present in a concentration from about 0.1% w/w to about 10% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the composition. In some embodiments, the nicotine component is present in a concentration from about 0.1% w/w to about 3% by weight, such as, e.g., from about 0.1% w/w to about 2.5%, from about 0.1% to about 2.0%, from about 0.1% to about 1.5%, or from about 0.1% to about 1% by weight, calculated as the free base and based on the total weight of the composition.

In some embodiments, the products or compositions of the disclosure can be characterized as free of any nicotine component (e.g., any embodiment as disclosed herein may be completely or substantially free of any nicotine component). By “substantially free” is meant that no nicotine has been intentionally added, beyond trace amounts that may be naturally present in e.g., a botanical material. For example, certain embodiments can be characterized as having less than 0.001% by weight of nicotine, or less than 0.0001%, or even 0% by weight of nicotine, calculated as the free base.

In some embodiments, the active ingredient comprises a nicotine component (e.g., any product or composition of the disclosure, in addition to comprising any active ingredient or combination of active ingredients as disclosed herein, may further comprise a nicotine component).

### Pharmaceutical Ingredients

In some embodiments, the active ingredient comprises an active pharmaceutical ingredient (API). The API can be any known agent adapted for therapeutic, prophylactic, or diagnostic use. These can include, for example, synthetic organic compounds, proteins and peptides, polysaccharides and other sugars, lipids, phospholipids, inorganic compounds (e.g., magnesium, selenium, zinc, nitrate), neurotransmitters or precursors thereof (e.g., serotonin, 5-hydroxytryptophan, oxitriptan, acetylcholine, dopamine, melatonin), and nucleic acid sequences, having therapeutic, prophylactic, or diagnostic activity. Non-limiting examples of APIs include analgesics and antipyretics (e.g., acetylsalicylic acid, acetaminophen, 3-(4-isobutylphenyl) propanoic acid), phosphatidylserine, myoinositol, docosahexaenoic acid (DHA, Omega-3), arachidonic acid (AA, Omega-6), S-adenosylmethionine (SAM), beta-hydroxy-betamethylbutyrate (HMB), citicoline (cytidine-5′-diphosphate-choline), and cotinine. In some embodiments, the active ingredient comprises citicoline. In some embodiments, the active ingredient is a combination of citicoline, caffeine, theanine, and ginseng. In some embodiments, the active ingredient comprises sunflower lecithin.

In some embodiments, the active ingredient is a combination of sunflower lecithin, caffeine, theanine, and ginseng.

The amount of API may vary. For example, when present, an API is typically at a concentration of from about 0.001% w/w to about 10% by weight, such as, e.g., from about 0.01%, about 0.02%, about 0.03%, about 0.04%, about 0.05%, about 0.06%, about 0.07%, about 0.08%, about 0.09%, about 0.1% w/w, about 0.2%, about 0.3%, about 0.4%, about 0.5% about 0.6%, about 0.7%, about 0.8%, about 0.9%, or about 1%, to about 2%, about 3%, about 4%, about 5%, about 6%, about 7%, about 8%, about 9%, or about 10% by weight, based on the total weight of the composition.

In some embodiments, the composition is substantially free of any API. By “substantially free of any API” means that the composition does not contain, and specifically excludes, the presence of any API as defined herein, such as any Food and Drug Administration (FDA) approved therapeutic agent intended to treat any medical condition.

### Sensory Additives

### Salts

In some embodiments, the composition comprises a salt (e.g., an alkali metal salt), typically employed in an amount sufficient to provide desired sensory attributes to the composition. Non-limiting examples of suitable salts include sodium chloride, potassium chloride, ammonium chloride, flour salt, sodium acetate, sodium citrate, calcium citrate, and the like. In some embodiments, the salt is sodium chloride, ammonium chloride, or a combination thereof. In some embodiments, the salt is trisodium citrate, calcium citrate, or a combination thereof.

When present, a representative amount of salt is about 0.1% by weight or more, about 0.5% by weight or more, about 1.0% by weight or more, or about 1.5% by weight or more, but will typically make up about 10% or less of the total weight of the composition, or about 7.5% or less, or about 5% or less (e.g., from about 0.1 to about 5% by weight or from about 0.5 to about 1.5%).

### Sweeteners

In order to improve the sensory properties of the composition according to the disclosure, one or more sweeteners may be added. The sweeteners can be any sweetener or combination of sweeteners, in natural or artificial form, or as a combination of natural and artificial sweeteners.

Examples of natural sweeteners include fructose, sucrose, glucose, maltose, mannose, galactose, lactose, isomaltulose, stevia, honey, and the like. Examples of artificial sweeteners include sucralose, maltodextrin, saccharin, aspartame, acesulfame K, neotame, and the like. In some embodiments, the sweetener comprises one or more sugar alcohols. Sugar alcohols are polyols derived from monosaccharides or disaccharides that have a partially or fully hydrogenated form.

Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). In some embodiments, the sweetener is sucralose, acesulfame K, or a combination thereof.

When present, a sweetener or combination of sweeteners may make up from about 0.01 to about 20% or more of the of the composition by weight, for example, from about 0.01 to about 0.1, from about 0.1 to about 1%, from about 1 to about 5%, from about 5 to about 10%, or from about 10 to about 20% by weight, based on the total weight of the composition. In some embodiments, a combination of sweeteners is present at a concentration of from about 0.01% to about 0.1% by weight of the composition, such as about 0.01, about 0.02, about 0.03, about 0.04, about 0.05, about 0.06, about 0.07, about 0.08, about 0.09, or about 0.1% by weight of the composition. In some embodiments, a combination of sweeteners is present at a concentration of from about 0.05% to about 0.5% by weight of the composition, such as about 0.1, about 0.2, about 0.3, about 0.4, or about 0.5% by weight of the composition. In some embodiments, a combination of sweeteners is present at a concentration of from about 1% to about 3% by weight of the composition.

### Flavouring Agents

In some embodiments, the composition comprises a flavouring agent. As used herein, a “flavouring agent,” “flavour” or “flavourant” is any flavourful or aromatic substance capable of altering the sensory characteristics associated with the oral product. Examples of sensory characteristics that can be modified by the flavouring agent include taste, mouthfeel, moistness, coolness/heat, and/or fragrance/aroma.

Flavouring agents may be natural or synthetic, and the character of the flavours imparted thereby may be described, without limitation, as fresh, sweet, herbal, confectionary, floral, fruity, or spicy. Specific types of flavours include, but are not limited to, vanilla, coffee, chocolate/cocoa, cream, mint, spearmint, menthol, peppermint, wintergreen, eucalyptus, lavender, cardamom, nutmeg, cinnamon, clove, cascarilla, sandalwood, honey, jasmine, ginger, anise, sage, licorice, lemon, orange, apple, peach, lime, cherry, strawberry, trigeminal sensates, terpenes, and any combinations thereof. See also, Leffingwell et al., Tobacco Flavoring for Smoking Products, R. J. Reynolds Tobacco Company (1972), which is incorporated herein by reference. Flavouring agents also may include components that are considered moistening, cooling or smoothening agents, such as eucalyptus. These flavours may be provided neat (i.e., alone) or in a composite, and may be employed as concentrates or flavour packages (e.g., spearmint and menthol, orange and cinnamon, lime, pineapple, and the like).

Representative types of components also are set forth in U.S. Pat. No. 5,387,416 to White et al.; US Pat. App. Pub. No. 2005/0244521 to Strickland et al.; and PCT Application Pub. No. WO 05/041699 to Quinter et al., each of which is incorporated herein by reference. In some instances, the flavouring agent may be provided in a spray-dried form or a liquid form.

The amount of flavouring agent utilized in the composition can vary, but is typically up to about 10% by weight, and certain embodiments are characterized by a flavouring agent content of at least about 0.1% by weight, such as about 0.5 to about 10%, about 1 to about 5%, or about 2 to about 4% weight, based on the total weight of the composition.

In some embodiments, the composition may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucolyptol, WS-3.

### Taste Modifiers

In order to improve the organoleptic properties of a composition as disclosed herein, the composition may include one or more taste modifying agents (“taste modifiers”) which may serve to mask, alter, block, or improve e.g., the flavour of a composition as described herein. Non-limiting examples of such taste modifiers include analgesic or anaesthetic herbs, spices, and flavours which produce a perceived cooling (e.g., menthol, eucalyptus, mint), warming (e.g., cinnamon), or painful (e.g., capsaicin) sensation. Certain taste modifiers fall into more than one overlapping category.

In some embodiments, the taste modifier modifies one or more of bitter, sweet, salty, or sour tastes. In some embodiments, the taste modifier targets pain receptors. In some embodiments, the composition may comprise a cannabinoid or other component having a bitter taste, and a taste modifier which masks or blocks the perception of the bitter taste. In some embodiments, the taste modifier is a substance which targets pain receptors (e.g., vanilloid receptors) in the user's mouth to mask e.g., a bitter taste of another component (e.g., a cannabinoid). Suitable taste modifiers include, but are not limited to, capsaicin, gamma-amino butyric acid (GABA), adenosine monophosphate (AMP), lactisole, or a combination thereof.

When present, a representative amount of taste modifier is about 0.01% by weight or more, about 0.1% by weight or more, or about 1.0% by weight or more, but will typically make up less than about 10% by weight of the total weight of the composition, (e.g., from about 0.01%, about 0.05%, about 0.1%, or about 0.5%, to about 1%, about 5%, or about 10% by weight of the total weight of the composition).

All percentages by weight described herein (denoted wt %) are calculated on a dry weight basis (DWB), unless explicitly stated otherwise. All weight ratios are also calculated on a dry weight basis. A weight quoted on a dry weight basis refers to the whole of the extract or slurry or material, other than the water or other solvent, and may include components which by themselves are liquid at room temperature and pressure, such as glycerol. Conversely, a weight percentage quoted on a wet weight basis (WWB) refers to all components, including water or other solvent.

For the avoidance of doubt, where in this specification the term “comprises” is used in defining the invention or features of the invention, embodiments are also disclosed in which the invention or feature can be defined using the terms “consists essentially of” or “consists of” in place of “comprises”. Reference to a material “comprising” certain features means that those features are included in, contained in, or held within the material.

The above embodiments are to be understood as illustrative examples of the invention. It is to be understood that any feature described in relation to any one embodiment may be used alone, or in combination with other features described, and may also be used in combination with one or more features of any other of the embodiments, or any combination of any other of the embodiments.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. A method for preparing a composition comprising one or more constituent, derivative or extract of cannabis in non-crystalline form, the method comprising extruding a precursor composition comprising one or more constituent, derivative or extract of cannabis.

2. A method as claimed in claim 1, wherein the extruded composition is soluble or dispersible in water.

3. A method as claimed in claim 1 or claim 2, wherein the one or more constituent, derivative or extract of cannabis in the extruded composition is soluble in water.

4. A method as claimed in any one of claims 1 to 3, wherein the constituent, derivative or extract of cannabis is one or more compounds selected from: cannabinoids; terpenes; alkaloids; and flavonoids.

5. A method as claimed in any one of claims 1 to 4, wherein the constituent, derivative or extract of cannabis is selected from the group consisting of: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabmolic acid (THCA), and tetrahydrocannabivarinic acid (THCV A).

6. A method as claimed in any one of claims 1 to 5, wherein the precursor composition comprises one or more acid form cannabinoid.

7. A method as claimed in claim 6, wherein the acid form cannabinoid is selected from the group consisting of cannabidiolic acid (CBDA), tetrahydrocannabinolic acid (THCA).

8. A method as claimed in any one of claims 1 to 7, wherein the precursor composition comprises one or more constituent, derivative or extract of cannabis in solution.

9. A method as claimed in any one of claims 1 to 7, wherein the precursor composition comprises one or more constituent, derivative or extract of cannabis in a colloid.

10. A method as claimed in any one of claims 1 to 7, wherein the precursor composition comprises one or more constituent, derivative or extract of cannabis in solid form.

11. A method as claimed in claim 10, wherein the precursor composition comprises the one or more constituent, derivative or extract of cannabis in crystalline form.

12. A method as claimed in any one of claims 1 to 11, wherein the extruded composition further comprises a water soluble material.

13. A method as claimed in claim 12, wherein the water soluble material is selected from the group consisting of: sugar alcohols; disaccharides; polysaccharides, such as cellulose, starch and their derivatives; dextrins; dextrates; natural gums; and polymers and copolymers, such as cellulosics.

14. A method as claimed in claim 13, wherein the water soluble material is selected from the group consisting of: mannitol, sorbitol, xylitol, isomalt, erythritol, arabitol, ribitol, maltitol, dulcitol, iditol and lactitol guar gum, acacia gum (also known as gum arabic), xanthan gum, locust bean gum, gellan gum, alginates and sodium alginates, hydroxypropyl methylcellulose (HPMC), hydroxypropyl methylcellulose acetate succinate (HPMCAS), polyvinylpyrrolidone (PVP), polyethylene glycol (PEG), polyethylene oxide (PEO), Macrogol 15 Hydroxystearate (Solutol HS 15®), and Vitamin E Polyethylene Glycol Succinate (Vit E TPGS).

15. A method as claimed in any one of claims 12 to 14, wherein the water soluble material is present in an amount of from about 10 to about 70% by weight, based on the total weight of the extruded composition.

16. A method as claimed in any one of claims 1 to 15, wherein the constituent, derivative or extract of cannabis is present in an amount of from about 0.1 to about 30% by weight, based on the total weight of the extruded composition.

17. A method as claimed in any one of claims 1 to 16, wherein the precursor composition comprises an additive which slows or inhibits crystallisation of the one or more cannabinoid in an aqueous environment.

18. A method as claimed in claim 19, wherein the additive is selected from the group consisting of polyvinylpyrrolidone (PVP), hydroxypropyl cellulose (HPC), hydroxypropyl methylcellulose (HPMC) and carboxymethyl cellulose (CMC).

19. A method as claimed in any one of claims 1 to 18, wherein the precursor composition further comprises a surfactant.

20. A method as claimed in claim 19, wherein the surfactant is selected from the group consisting of: glyceryl monooleate; and sodium lauryl sulfate (sodium dodecyl sulfate, SLS, or SDS), docusate sodium, lecithin, polyoxyethylene sorbitan fatty acid esters (Polysorbate, Tween®), polyoxyethylene 15 hydroxy stearate (Macrogol 15 hydroxy stearate, Solutol HS15®), polyoxyethylene castor oil derivatives (Cremophor® EL, ELP, RH 40), polyoxyethylene stearates (Myrj®), sorbitan fatty acid esters (Span®), polyoxyethylene alkyl ethers (Brij®), polyoxyethylene nonylphenol ether (Nonoxynol®), sugar esters and lecithins.

21. A method as claimed in claim 19 or claim 20, wherein the precursor composition comprises surfactant in an amount of from about 0.5 to about 10% by weight, based on the total weight of the composition.

22. A method as claimed in any one of claims 1 to 21, wherein the extruded composition further comprises a disintegrant selected from the group consisting of: croscarmellose, sodium starch glycolate, and crospovidone, povidone (PVP); and the like.

23. A method as claimed in any one of claims 1 to 22, wherein the extruded composition further comprises an effervescent agent or combination of agents.

24. A method as claimed in any one of claims 1 to 23, wherein the extruded composition comprises a flavour or sensate.

25. A method as claimed in any one of claims 1 to 24, wherein the extruded composition comprises a further active agent.

26. A method as claimed in any one of claims 1 to 25, the precursor composition comprising a cannabis plant extract.

27. A method as claimed in claim 26, wherein the cannabis plant is hemp.

28. A method as claimed in any one of claims 1 to 27, wherein the extruded composition is formed into a solid unit dosage form, a powder or granules.

29. A method as claimed claim 28, wherein the extruded composition is formed into particles having a volume mean particle size of from about 50 μm to about 500 μm.

30. A method as claimed in claim 28 or claim 29, comprising cutting and/or spheronisation of the extruded composition.

31. An extruded composition obtained or obtainable by a method as claimed in any one of claims 1 to 30.

32. An oral product for providing buccal delivery of one or more constituent, derivative or extract of cannabis, comprising a composition obtained or obtainable by a method as claimed in any one of claims 1 to 30.

33. An oral product as claimed in claim 32, wherein the release of one or more constituent, derivative or extract of cannabis from the oral product begins within a period of 5, 10, 15 or 30 seconds following exposure to the aqueous environment of the oral cavity.

34. An oral product as claimed in claim 32 or 33, wherein the one or more constituent, derivative or extract of cannabis is released from the oral product over a period of at least 5, 10, 15, 20, 25, 30, 45 or 60 minutes following exposure to the aqueous environment of the oral cavity.

35. An oral product as claimed in any one of claims 32 to 34, comprising a pouch containing the extruded composition.

36. An oral product as claimed in any one of claims 32 to 35, comprising a dissolving strip comprising the extruded composition.

37. An oral product as claimed in claim 36, the dissolving strip comprising a bioadhesive.
